(19) United States
(12) Patent Application Publication
(10) Pub. No.: US 2004/0096427 A1
(43) Pub. Date: May 20, 2004

(54) Oral gram(+) bacteria and glutamine composition for prevention and/or treatment of gastro-intestinal dysfunctions including inflammation in the gastro-intestinal tract, neonatal necrotizing enterocolitis (nec) and bacterial sepsis

(76) Inventor: Pinaki Panigrahi, Columbia, MD (US)

Correspondence Address: Piper Rudnick, Supervisor Patent Prosecution Services, 1200 Nineteenth Street NW, Washington, DC, 20036-2412, US

(21) Appl. No.: 10/275,171

(22) PCT Filed: May 3, 2001
(86) PCT No.: PCT/US01/14080

(51) Int. Cl. A61K 45/00; A61K 31/198

(52) U.S. Cl. 424/93.4; 514/563

(57) ABSTRACT

A composition and method for treating and preventing gastro-intestinal injury are disclosed. The composition includes a combination of Gram (+) bacteria, in particular Lactobacillus and/or Bifidobacteria, and glutamine. The method involves orally or naso-gastrically administering a composition containing Gram (+) bacteria, in particular Lactobacillus, and glutamine. The composition, which blocks translocation of bacterial agents such as Gram (−) bacteria, other infectious agents, toxins, chemicals and injurious substances, may be used in the prevention and treatment of symptoms and/or disease that result from translocation of Gram (−) bacteria, including inflammation in the gastro-intestinal tract, Neonatal Necrotizing Enterocolitis (NEC) and bacterial sepsis.

This application claims priority to U.S. Provisional Application Serial No. 60/201,408, filed May 3, 2000, the entire contents of which are incorporated herein by reference.

### BACKGROUND OF THE INVENTION

1. Field of the Invention

The present invention relates to the use of Gram (+) bacteria, and in particular Lactobacillus and/or Bifidobacteria, and luminal glutamine in combination to prevent and/or treat gastro-intestinal dysfunction. The combination of Gram (+) bacteria and glutamine of the invention, which prevent translocation of Gram (−) bacteria across mucosal layers, can be used to protect intestinal cells against injury caused by disease, infectious agents, toxins, chemicals and other injurious substances. The combination of Gram (+) bacteria and glutamine of the invention can be used, in particular, to prevent and/or treat symptoms and/or disease that result from translocation of Gram (−) bacteria across mucosal layers, including inflammation in the gastrointestinal tract, Neonatal Necrotizing Enterocolitis (NEC) and bacterial sepsis.

2. Background of the Prior Art

Digestive problems, which comprise the number one health problem in North America, appear to be occurring with more frequency in recent years. One way to maintain digestive health is to maintain proper intestinal flora. Bacterial translocation, i.e., the passage of a few viable intestinal bacteria across the intestinal epithelial cell layer into the normally sterile extra intestinal tissues is a normal process. The mucosal immune system (macrophages as first line of defense) along with the consequent immune activation generally prevent detrimental translocation. Secretory immunoglobulins may also prevent the attachment of detrimental bacteria to the mucosal surface. Bacterial translocation has been suggested to play a role in the etiology of posttraumatic infections and multiple organ failure. This is presumed to be due to a breakdown of the intestinal mucosal barrier, which in turn permits pathogenic bacteria to pass into the blood stream.

Sepsis is a serious clinical condition in which infective agents such as pathogenic bacteria, or products of infection such as toxins, enter the blood circulation and profoundly affect a patient's blood pressure, heart rate and body temperature. Sepsis, which can originate anywhere in the body, including the gastro-intestinal tract, is often treated by administering intravenous antibiotics.

Drug companies are investigating several promising agents to treat sepsis, but no cure has emerged yet. In the meantime, sepsis is on the rise because more pathogens are becoming antibiotic-resistant, and more people are at risk than ever before because of advanced age and multiple medical problems.

In 65% to 70% of cases, sepsis is caused by Gram (−) pathogens such as E. coli and the Pseudomonas and Klebsiella species. These pathogens injure tissue, triggering an inflammatory reaction that signals the immune system to destroy pathogens and contain the infection. The immune system releases numerous chemical mediators that kill pathogens, disable toxins, and alert the central nervous system and white blood cells. The mediators also dilate blood vessels, improving blood flow to the injured area, and increase capillary permeability, delivering more white blood cells to the area. The clotting cascade is activated to isolate the area and help contain the infection.

As pathogens are destroyed, they release toxins that injure the capillaries' endothelium and further increase capillary permeability.

If the infection is severe, the immune response escalates. The toxins and chemical mediators circulating in the blood cause peripheral and pulmonary edema, hemodynamic instability, and malfunctions in oxygen transport. About two-thirds of sepsis-related deaths are caused by refractory hypotension from vasodilation or decreased cardiac output.

Sepsis involves a very complex sequence of events and much work still needs to be done to completely understand how a patient goes into septic shock. Patients with septic shock have a biphasic immunological response. Initially they manifest an overwhelming inflammatory response to the infection. This is most likely due to the pro-inflammatory cytokines Tumor Necrosis Factor (TNF), IL-1, IL-12, Interferon gamma (IFNgamma) and IL-6.

The body then regulates this response by producing anti-inflammatory cytokines (IL-10), soluble inhibitors (TNF receptors, IL-1 receptor type II and IL-1RA (an inactive form of IL-1)), which is manifested in the patient by a period of immunodepression. Persistence of this hyporesponsiveness is associated with increased risk of nosocomial infection and death.

The pro-inflammatory cytokines produced are tumor necrosis factor (TNF), Interleukins 1, 6 and 12 and Interferon gamma (IFNgamma). These cytokines can act directly to affect organ function or they may act indirectly through secondary mediators. The secondary mediators include nitric oxide, thromboxanes, leukotrienes, platelet-activating factor, prostaglandins and complement.

Then these primary and secondary mediators cause the activation of the coagulation cascade, the complement cascade and the production of prostaglandins and leukotrienes. Endothelial cell damage occurs which affects profusion of the organs and can lead to multiple organ system failure.

The use of antibiotics has a profound effect on the normal flora and can result in colonization with antibiotic-resistant organisms. Antibiotic-mediated disruption of the normal flora can thus lead to infection and its sequele.

Like many groups of living things, bacteria have “friendly” and “unfriendly” populations. Friendly bacteria play a major role in balancing and counteracting the unfriendly bacteria. When friendly bacteria are not at appropriate levels and when unfriendly bacteria dominate the intestinal flora, health problems such as described above can result.

Lactobacilli are one of the most important types of friendly bacteria found in the digestive tract. The bacteria, which are named because they are able to turn sugar into lactic acid, play a key role in producing fermented milk, yogurt and cheese. In the early 1900's, Elie Metchinkoff hypothesized that Lactobacilli would provide a hostile environment to unfriendly bacteria in the intestinal environment. This hypothesis was later proven correct. Lactobacilli and Bifidobacteria, another “friendly bacteria” found in the digestive tract, have long been known to have positive effects in the intestine especially in maintaining a healthy gut microflora. These organisms generally act when they are available at the action site (intestine) live to exert their effects. These organisms are also known to secrete antimicrobial substances known as bacteriocins, i.e., substances that kill closely-related strains of other bacteria.

Lactobacilli and Bifidobacteria are known to prevent pathogenic microorganisms from colonizing on body surfaces (colonization resistance). Commercial preparations of Lactobacilli and/or Bifidobacteria have thus been used to restore normal intestinal flora after imbalance created by antibiotic therapy. A variety of in vitro studies also indicate that additional endogenous intestinal bacteria can inhibit pathogenic bacteria. For example, Sullivan et al., Inhibitions of growth of C. botulinum by intestinal microflora isolated from healthy infants, Microbial. Ecology in Health and Disease, 1:179-192 (1988), showed that in addition to Bifidobacteria and Lactobacilli, gut isolates of Proprionibacteria and Enterococci inhibit C. botulinum in vitro.

Despite significant advances in recent neonatal practice, neonatal necrotizing enterocolitis (NEC) remains a major cause of mortality in premature infants. Survivors of NEC have considerable long-term morbidity resulting from the disease, including short-gut syndrome, failure to thrive, intestinal stricture and the need for repeated surgery. Although 11% of premature infants born weighing less than 1500 g develop NEC, the cause of the disease remains unclear and no specific treatments are available. A reasonable hypothesis suggests that a combination of factors including prematurity, intestinal ischemia and bacterial colonization lead to stimulation of an inflammatory cascade and a resulting final common pathway of NEC.

Bacterial colonization of the neonatal gastro-intestinal tract begins when the infant encounters maternal cervical and vaginal bacteria during delivery. Brooke et al., Aerobic and anaerobic bacterial flora of the maternal cervix and newborn gastric fluid and conjunctiva: A prospective study, Pediatrics, 63:451-455 (1979). By 10 days of age, the majority of healthy full-term newborns are fully colonized with a variety of bacterial species. Lone et al., Development of anaerobic fecal flora in healthy newborn infants, J. Pediatr., 91:298-301 (1977). The gut of a premature infant is not colonized with the normally heterogeneous bacterial flora and instead demonstrates delayed colonization with only a limited number of bacterial species. Gupta et al., Endemic necrotizing enterocolitis: lack of association with a specific infectious agent, Pediatr. Infect. Dis., 13:725-734 (1994). It has been shown that the stool of preterm infants, with and without NEC, is colonized on the average by fewer than 2.5 species of aerobic bacteria, compared to >10 species in full terms. Gupta et al. (1994). It is believed that limited friendly bacterial colonization at least in part permits pathogenic bacterial overgrowth that could in turn initiate the cascade of events that lead to NEC.

The present inventors have observed that translocation of Gram (−) bacteria across mucosal layers causes adverse symptoms and/or disease, including inflammation in the gastrointestinal tract, NEC and bacterial sepsis. The present inventors have also observed that certain Gram (+) bacteria prevent translocation of pathogenic Gram (−) bacteria across mucosal layers. The present inventors have thus demonstrated a causal link between Gram (−) bacterial translocation and subsequent development of symptoms and/or disease such as inflammation in the gastro-intestinal tract, NEC and bacterial sepsis.

The present inventors have observed that it is not the species or strain of bacteria that is responsible for evoking an injury or response, but rather the microbial ecology (i.e., the combination of Gram (−) and Gram (+) bacteria) that either protects or gives rise to symptoms and/or disease, including inflammation in the gastro-intestinal tract, NEC and bacterial sepsis. Based on this, the inventors have developed compositions and methods to modify the microbial flora of the intestine in such a way to block (or minimize) the deleterious effects of Gram (−) bacteria. The compositions and methods prevent bacterial translocation and symptoms and/or disease, including inflammation in the gastro-intestinal tract, NEC and bacterial sepsis.

U.S. Pat. No. 5,981,590 to Panigrahi et al., the relevant portions of which are incorporated herein by reference, discloses a method of treating and/or preventing necrotizing tissue injury in the gastro-intestinal tract by using oral glutamine. In accordance with-the method, supply of glutamine from the luminal side (apically for the enterocytes) has been shown to reduce bacterial translocation and maintain healthy physiological functions. Prior to the method, beneficial effects of glutamine had been shown only by parenteral (intravenous route) administration, and the effects were described to be mediated via multiple immunological functions. U.S. Pat. No. 5,981,890 teaches that supply of glutamine to the enterocytes from the apical side (luminal) is important in the maintenance of physiological functions. Lack of such apical glutamine results in decreased transepithelial resistance, increased passage of inulin, and increased bacterial translocation of pathogenic organisms across intestinal cell monolayers. Translating the effects in vivo, via instillation of rabbit ileal loops, which are often used as a model, with glutamine protects them against Gram (−) bacteria-induced necrotizing enterocolitis.

U.S. Pat. No. 6,132,710 to Panigrahi et al., the relevant portions of which are incorporated herein by reference, discloses that two strains of Lactobacillus, i.e., Lactobacillus acidophilus and Lactobacillus plantarum, were capable of blocking the adherence and translocation of Gram (−) organisms such as E. coli in an in vitro system (Caco-2 cell culture model) and reduced tissue injury and inflammatory cell infiltration in a rabbit model (ileal loop model), suggesting that they are useful in treatment and/or prevention of NEC.

Although the clinical use of Gram (+) bacteria such as Lactobacillus and Bifidobacteria and the use of glutamine to enhance intestinal defense against potential luminal pathogens have individually been tested in vitro and in vivo, there remains a need for further investigation and development in the fight against gastro-intestinal dysfunctions and resulting adverse symptoms and/or disease.

### SUMMARY OF THE INVENTION

The present invention relates to the use of Gram (+) bacteria, and in particular Lactobacillus and/or Bifidobacteria, and luminal glutamine in combination to protect intestinal cells against injury caused by disease, infectious agents, toxins, chemicals and other injurious substances. The combination of Gram (+) bacteria and glutamine of the invention can be used to prevent and/or treat gastro-intestinal dysfunctions and resulting adverse symptoms and/or disease, including inflammation in the gastro-intestinal tract, Neonatal NEC and bacterial sepsis

The Gram (+) bacteria and glutamine composition of the present invention may thus be used to prevent and/or treat diseases of the gastrointestinal tract that may have a bacterial etiologic component. For example, the Gram (+) bacteria and glutamine composition of the present invention may be used to treat full-terms, children, and adults, in gastrointestinal dysfunctions of infective and/or inflammatory origin where bacterial infection may act as a trigger or aid in disease progression.

The Gram (+) bacteria and glutamine composition of the present invention may be used to treat both pediatric and adult patients, especially those in intensive care units under total parenteral nutrition (intravenous feed) to avoid mucosal dysfunction and further bacterial translocation.

The Gram (+) bacteria and glutamine composition of the present invention may also be used to treat patients undergoing chemotherapy, irradiation and bone marrow transplantation.

The Gram (+) bacteria and glutamine composition of the present invention may be used to prevent and treat food allergy and intolerance, where injury caused by an antecedent bacterial infection allows the passage of food antigens through the gut mucosa and further triggers the inflammatory process.

The Gram (+) bacteria and glutamine composition of the present invention may also be used to prevent and/or treat other gastro-intestinal disorders including but not limited to Celiac disease, where initial damage to the gut mucosa allows the passage of the triggering antigen to gain access to deeper layers of the intestine, which in turn in concert with other immunologic, infective or genetic factors can cause the clinical disease.

The Gram (+) bacteria and glutamine composition of the present invention would be particularly useful to prevent and/or treat Neonatal Necrotizing Enterocolitis and bacterial sepsis.

A preferred method of treating gastrointestinal dysfunctions such as discussed above includes orally (or naso-gastrically) administering an effective amount of the Gram (+) bacteria and glutamine composition for improving gastro-intestinal physiological functions.

The Grain (+) bacteria and glutamine composition may be administered in any form that is orally (or naso-gastrically) administrable, including powder forms or in a reconstituted mixture with a fluid. Alternatively, the Gram (+) bacteria and glutamine composition may be administered in a capsule. Preferably, the capsules are acid resistant slow-release micro-capsules that last long enough to reach the requisite areas in the gastro-intestinal tract.

These and further features of the invention are apparent in the disclosure, which includes the above and ongoing written description, the claims and the drawings.

### BRIEF DESCRIPTION OF THE DRAWINGS

FIG. 1a shows the amount of E. coli in the blood of ileal loop subjects at 3 hours, 6 hours and 12 hours, respectively, after inoculation of the gut. FIG. 1b shows the amount of E. coli in the blood of ileal loop subjects at 3 hours, 6 hours and 12 hours, respectively, after exposure of the gut to a Lactobacillus and glutamine composition.

FIG. 2 shows the effect of glutamine and Lactobacillus on E. coli translocation at 1 hour after treatment. D: deprived of glutamine; D/R: deprived of glutamine then treated with glutamine alone; D/PP217: deprived of glutamine then treated with Lactobacillus strain ATCC 202195; D/R/P: deprived of glutamine then treated with a Lactobacillus strain ATCC 202195 and glutamine composition.

FIG. 3 shows the effect of glutamine and Lactobacillus on E. coli translocation at 3 hours after treatment. D: deprived of glutamine; D/R: deprived of glutamine then treated with glutamine alone; D/PP217: deprived of glutamine then treated with Lactobacillus strain ATCC 202195; D/R/PP217: deprived of glutamine then treated with a Lactobacillus strain ATCC 202195 and glutamine composition.

FIG. 4 shows the effect of glutamine and Lactobacillus on E. coli translocation at 6 hours after treatment. D: deprived of glutamine; D/R: deprived of glutamine then treated with glutamine alone; D/PP217: deprived of glutamine then treated with Lactobacillus strain ATCC 202195; D/R/P: deprived of glutamine then treated with a Lactobacillus strain ATCC 202195 and glutamine composition.

### DESCRIPTION OF THE PRESENT INVENTION

As discussed above, it has previously been shown that adherent Gram (−) bacteria, e.g. E. coli can cause NEC-like and sepsis-like injury in a rabbit ileal loop model, that such injury appears to be caused by translocation of the Gram (−) bacteria across mucosal layers, and that Gram (+) bacteria and glutamine individually can block such injury.

### Use of Lactobacillus and Glutamine in Treating NEC

In the present invention, a Lactobacillus and glutamine composition is shown to have a synergistic and total disease blocking ability against NEC. Tissue culture previously used to examine the effects of Lactobacillus and glutamine alone were used to examine the effects of the combination. It is expected that animal models previously used to examine the effects of Lactobacillus and glutamine alone would provide additional evidence of the synergistic effects of the Lactobacillus and glutamine composition.

The following examples are provided for illustrative purposes only and are in no way intended to limit the scope of the present invention.

### EXAMPLES

### Caco-2 Cell Culture System

Caco-2 cells derived from human adenocarcinoma cells which show all the morphological and functional characteristics of mature small intestinal epithelial cells after differentiation were employed in a number of experimental systems. Panigrahi et al., Development of an in vitro model for study of non-01 Vibrio cholerae virulence using Caco-2 cells, Infect. Immun., 58:3415-3424 (1990). Caco-2 cells were grown in DMEM supplemented with 1% nonessential amino acids, 1% sodium pyruvate, 10% fetal calf serum, 100 U penicillin and 100 μg of streptomycin/mL in a 5% CO₂ atmosphere at 37° C. For transcytosis studies, 0.2×10⁶ cells in 0.3 mL medium were seeded on the apical side of 0.6 cm² polycarbonate transwell filters/clusters (Costar, Cambridge, Mass.). Each basolateral chamber received 1 mL of medium, which was changed every third day.

### Caco-2 Cell Transwell System

A Caco-2 cell transwell system was used in accordance with Panigrahi et al. (1990) and Panigrahi et al. (1994) to grow cells on a membrane allowing the measurement of bacteria that translocate. Briefly, Caco-2 cells were grown on polycarbonate filters in transwell cluster and TEER (trans-epithelial electrical resistance) was measured before and after treatment (1) with E. coli alone, (2) in a system deprived of glutamine, (3) in a system deprived of glutamine and later replenished with glutamine, and (4) in a system deprived of glutamine and treated with a Lactobacillus strain ATCC 202195 and glutamine composition.

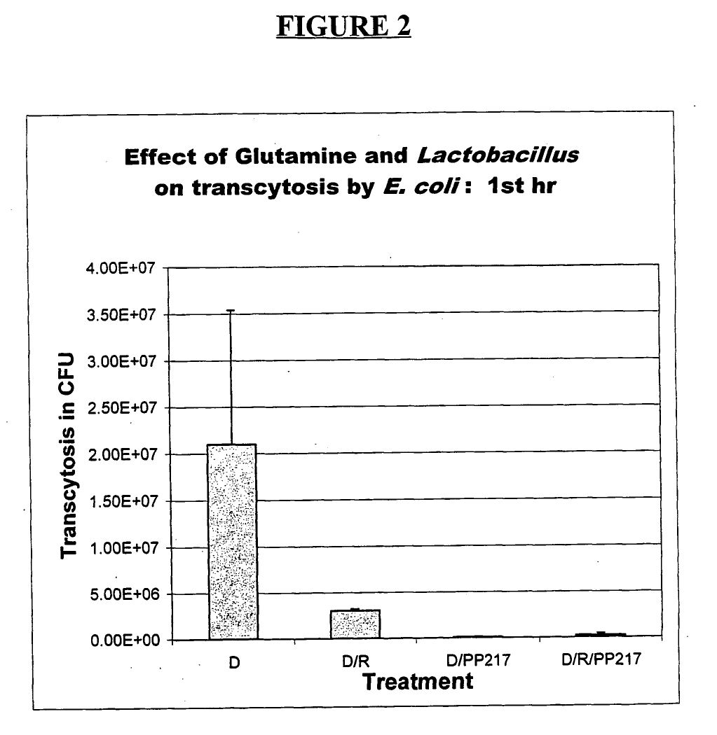

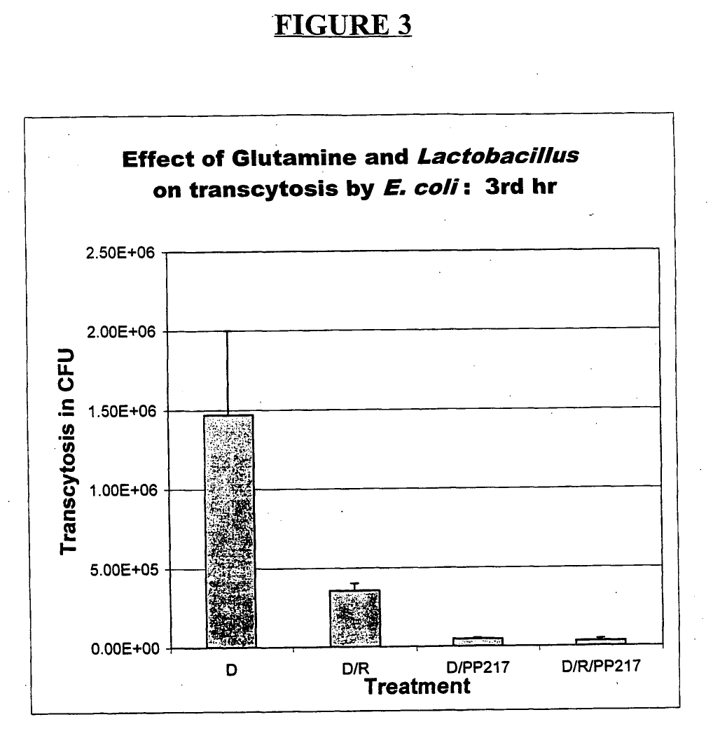

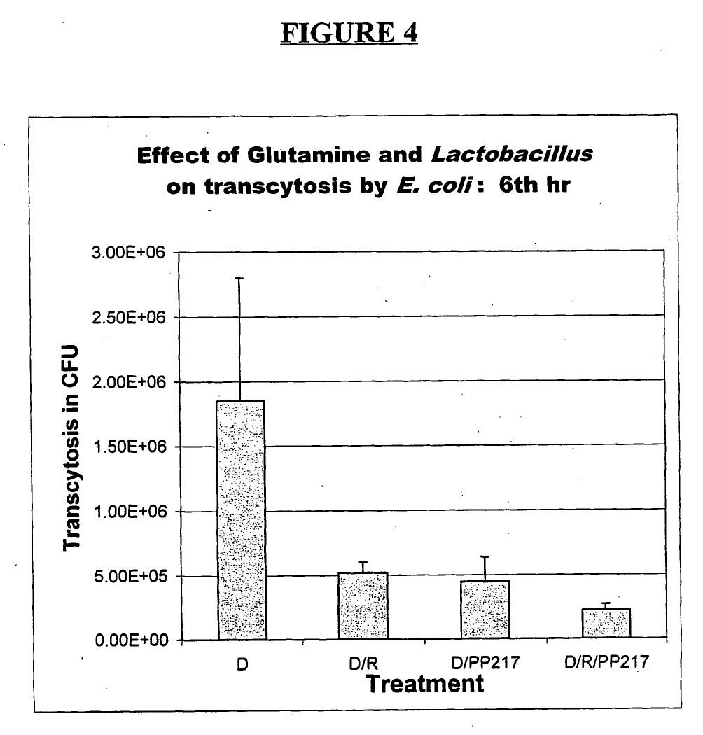

While there was no discernible difference between the individual components and the combination during 1 and 3 hours, there was a significant difference when the experiment was extended to the sixth hour. FIGS. 2, 3 and 4 describe the phenomenon. It is now conceived that if experiments could be done over a longer term (which is difficult with cultured cells after bacterial infection), a bigger difference would be seen with the Lactobacillus and glutamine composition. It is also conceived that since animal experiments can be done over a longer term, e.g., 12-24 hours, the same or better effects of the Lactobacillus and glutamine composition in the rabbit ileal loop model would be observed.

### In Vivo Weanling Rabbit Ileal Loop Studies

Following previously described protocols, (Panigrahi P. Gupta S. Gewolb I H. and Morris J G Jr., Occurrence of Necrotizing Enterocolitis may be dependent on patterns of bacterial adherence and intestinal colonization: Studies in Caco-2 tissue culture and weanling rabbit models. Ped. Res. 36 (1):115-121 (1994)), weanling rabbit ileal loop model were used to determine the effects of the Lactobacillus and glutamine composition in vivo as follows: weanling rabbit ileal loops were infected with either (1) E. coli alone (10⁸ organisms/ml) or (2) E. coli and the Lactobacillus and glutamine composition, wherein, 10⁸⁻¹² organisms/ml of Lactobacillus and 1-4 mM glutamine were used. Typical NEC-like disease symptoms were observed in E. coli controls and total protection against E. coli induced damage was observed in-the loops receiving the Lactobacillus and glutamine composition. Although the ileal loop experiment exhibits what would happen in a real-life situation, it is expected that the amount of Lactobacillus and/or the amount of glutamine necessary for treatment in a real-life situation will vary depending on the needs of the patient. For example, a higher Gram (−) bacterial load is normally observed in a real-life situation. Therefore, a higher load of Lactobacillus would be required. It is also expected that since a real-life situation develops over days, the Lactobacillus and glutamine composition will produce even more pronounced effects than that shown in the rabbit ileal loop model.

### Anti-Inflammatory Effects of Lactobacillus and Glutamine Composition

The anti-inflammatory effects of a Lactobacillus and glutamine composition can be determined in rabbit ileal loop experiments by RT-PCR in accordance with previously described methods.

### Use of Lactobacillus and Glutamine in Treatment of Bacterial Sepsis

Bacterial sepsis in neonates is generally considered to be initiated by entry of bacteria from the skin into the blood stream via different central intravenous lines. It is estimated that similar events occur in pediatric and adult populations. The bigger picture of sepsis is rather complicated and the exact mechanisms are not known.

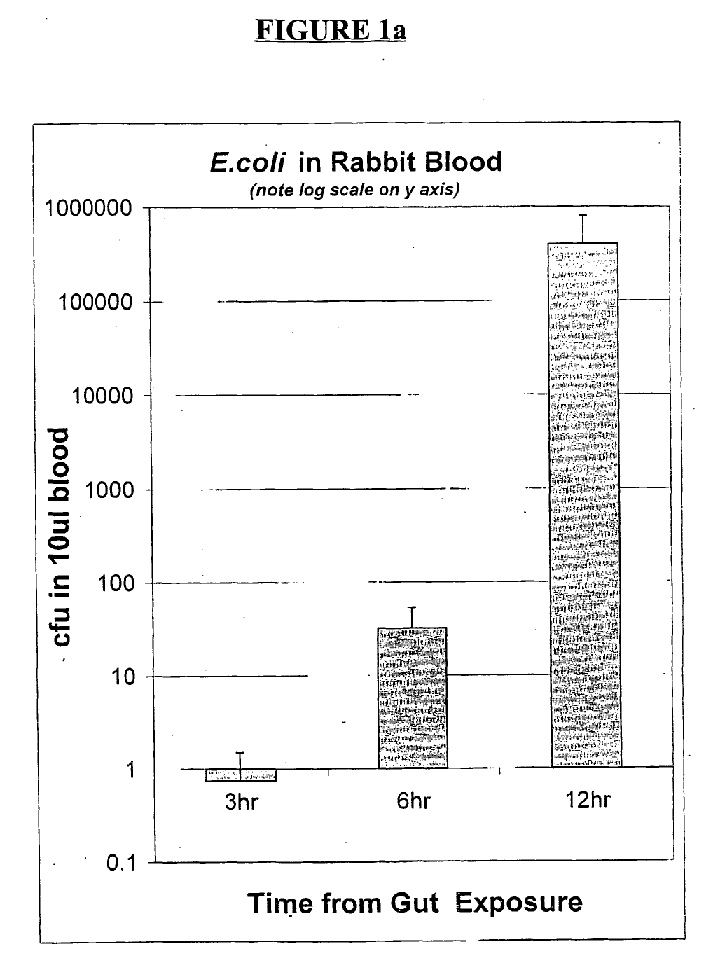

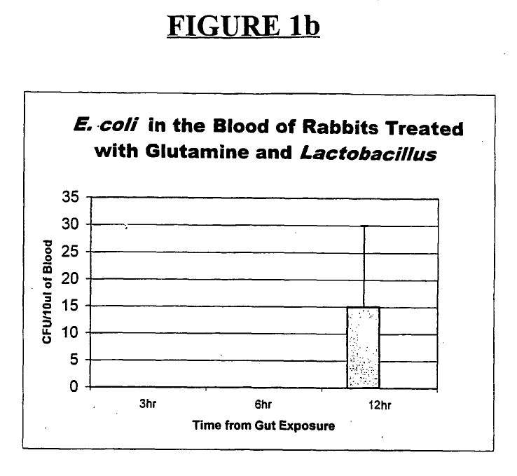

The present inventors have observed that all of the rabbits that weighed less than 400 gm developed some degree of sepsis after being infected with E. coli bacteria alone (10⁸ organisms/ml) in the ileal loops. A small number of E. coli bacteria could be cultured from blood during the first hours, but the number increased in logarithmic scale as time progressed (see FIG. 1a), and at the time of sacrifice and necropsy, very high numbers of bacteria were cultured. When a Lactobacillus and glutamine composition was used, there was total blockage of sepsis during the first six hours (see FIG. 1b). Note the more than 100,000 E. coli in the blood of control animals, comparable to severe sepsis. In rabbits treated with a combination of Lactobacillus and glutamine, there were only 10-25 (mean of 15) bacteria in the blood only late at 12 hr. (no bacteria at 3 or 6 hr.). This is a very small number and can probably be handled easily by the gut defense mechanisms (macrophages, etc.). For the reasons discussed above, it is expected that a Lactobacillus and glutamine composition would have an even more pronounced effect in treating and/or preventing sepsis for more extended periods of time.

As a result of extensive investigation, the present inventors have determined that Lactobacillus and glutamine exert their primary effects on the gastro-intestinal physiological function differently. The present inventors have determined:

1. That Lactobacillus prevents initial steps of E. coli (and therefore Gram (−) bacteria) adherence to gastro-intestinal mucosa and affects immunological responses that follow.

2. That glutamine does not have an effect on E. coli (and therefore Gram (−) bacteria) adherence to gastro-intestinal mucosa.

3. That glutamine prevents translocation of E. coli (and therefore Gram (−) bacteria) across mucosal layers and the downstream injurious effects.

4. That a combination of adherence and translocation is required in order for E. coli, and other Gram (−) bacteria, to exert their adverse effects. (The bacteria has to attach to the intestinal mucosa before passing through.)

5. That, while each of Lactobacillus and glutamine individually show beneficial effects over a short period of time, a combination of Lactobacillus and glutamine shows beneficial effects long term.

6. That a Lactobacillus and glutamine composition, which prevents both E. coli adherence and E. coli translocation, is an almost total and unprecedented blockage of adverse effects caused by E. coli (and Gram (−) bacteria) and is therefore an almost total and unprecedented blockage of disease.

7. That the Lactobacillus and glutamine composition can negate effects of different types of bacteria. For example, some bacteria are more invasive than others, i.e., some bacteria pass through the intestinal mucosa more readily, wherein other less invasive bacteria exert their adverse effects due to large numbers present. The Lactobacillus and glutamine composition can nail down both aspects, and thus provide a synergystic effect.

8. That, surprisingly, the Lactobacillus and glutamine composition has even a more significant effect, and is able to protect against massive dose infections.

Thus, the Lactobacillus (and more specifically Lactobacillus strain ATCC 202195) and glutamine composition can prevent and/or treat gastro-intestinal dysfunction, NEC and bacterial sepsis. When given in a pharmaceutically acceptable composition and in therapeutic dosages, the two agents in combination block bacterial transcytosis in a transwell cluster system and, are expected to block NEC-like disease in a weanling rabbit model and in a real-life situation. The two agents combined thus exhibit synergistic effects for improved treatment and long-term protection.

Various other modifications will be apparent to and can be readily made by those skilled in the art without departing from the scope and spirit of this invention. Accordingly, it is not intended that the scope of the claims appended hereto be limited to the description as set forth herein, but rather that the claims be broadly construed.

What is claimed is:

1. A method of preventing gastro-intestinal dysfunction comprising orally administering a Gram (+) bacteria and glutamine composition.

2. The method of claim 1, wherein said Gram (+) bacteria and glutamine composition protects tissues along the gastro-intestinal tract by blocking bacteria or other agent adherence to mucosal layers and by blocking bacteria or other agent translocation across said mucosal layers.

3. The method of claim 2, wherein translocation of agents selected from the group consisting of infectious agents, toxins, chemicals, and injurious substances is blocked.

4. The method of claim 1, further comprising optimizing mucosal defense due to an intraluminal/apical presence of the Gram (+) bacteria and glutamine composition.

5. The method of claim 1 wherein the administering comprises administering to individuals selected from a group consisting of pre-term infants, full-term infants, children and adults.

6. The method of claim 1 wherein the administering comprises administering the Gram (+) bacteria and glutamine composition as a powder.

7. The method of claim 1, wherein the administering comprises administering the Gram (+) bacteria and glutamine composition mixed in a fluid.

8. The method of claim 1, wherein the administering comprises administering the Gram (+) bacteria and glutamine composition as capsules.

9. The method of claim 8, wherein the capsules are acid-resistant slow-release micro-capsules.

10. The method of claim 8, wherein the capsules are coated acid-resistant slow-release capsules.

11. The method of claim 1, further comprising administering other drugs for treatment of gastro-intestinal ailments with the Gram (+) bacteria and glutamine composition.

12. The method of claim 2, wherein the blocking bacterial translocation comprises blocking Gram (−) bacteria.

13. A method of preventing gastro-intestinal dysfunction characterized by infection or inflamation comprising orally administering a Gram (+) bacteria and glutamine composition and allowing the Gram (+) bacteria and glutamine composition to coat gastro-intestinal mucosa.

14. The method of claim 13, further comprising preventing conditions selected from the group consisting of NEC and bacterial sepsis.

15. A Gram (+) bacteria and glutamine composition.

16. The Gram (+) bacteria and glutamine composition of claim 15, said Gram (+) bacteria and glutamine composition blocks epithelial cell bacterial adherence and translocation.

17. A vaccine prepared using the Gram (+) bacteria and glutamine composition of claim 15.
